(19) United States
(12) Patent Application Publication
(10) Pub. No.: US 2005/0119573 A1
(43) Pub. Date: Jun. 2, 2005

(54) Method and system for quantification of arterial stenosis

(76) Inventors: Boris Vilenkin, Ontario (CA); Margarita Vilenkin, Ontario (CA)

Correspondence Address: MARK D. SARALINO (GENERAL);RENNER, OTTO, BOISELLE & SKLAR, LLP, 1621 EUCLID AVENUE, NINETEENTH FLOOR, CLEVELAND, OH, 44115-2191, US

(21) Appl. No.: 10/982,328

(22) Filed: Nov. 5, 2004

Related U.S. Application Data
(60) Provisional application No. 60/517,482, filed on Nov. 5, 2003.

(51) Int. Cl. A61B 8/06

(52) U.S. Cl. 600/450

(57) ABSTRACT

A method is proposed that identifies and quantifies stenoses in arteries based on an analysis of Doppler frequency shifts from several heartbeats. It is non-invasive and individual insensitive. Pulsatile flow through a blood vessel with wall roughness and/or variable lumen area generates flow disturbances, which lead to variations in the shape of the Doppler shift frequency spectrum. One or several frequency bands that are affected by these flow disturbances are selected from the overall Doppler shift frequency spectrum. Next, one or several parameters, which characterize the selected frequency bands and vary with the degree of stenosis, are used in a linear function to calculate the percentage of lumen area reduction. This method applies in the clinically important range of lumen area reduction of 10-70% with a standard error of 5% or less. For lumen area reductions greater than about 70%, the standard error is larger. A system for practical implementation is also proposed.

### RELATED APPLICATION DATA

This application claims priority of U.S. Provisional Application No. 60/517,482 filed on Nov. 5, 2003, which is incorporated herein by reference in its entirety.

### FIELD OF THE INVENTION

The present invention relates generally to vascular diagnostic techniques and, more particularly, to a non-invasive, individual-insensitive system and method for quantification of arterial stenosis.

### BACKGROUND OF THE INVENTION

Arteriosclerosis is a chronic disease characterized by abnormal thickening of the arterial walls due to lipid deposition and subsequent accretion of platelets and other cells forming plaque. This process is accompanied by arterial lumen narrowing. From a medical point of view, arteriosclerosis affecting the coronary arteries and carotid arteries is of most concern. Atherosclerotic stenoses prevent normal blood supply to the heart and brain and is a major cause of preventable morbidity, disability and premature death in developed countries. As a result, periodic screening of patients to detect and assess obstructive coronary and carotid lesions is vital for the diagnosis, treatment and prevention of disease. Arterial wall thickening depends primarily on the deposit of low density lipids (LDL) on the inner surface of a vessel wall. This deposition begins soon after birth in the form of lipid stripes and continues throughout life. The lipid stripes appear in those parts of the arterial tree where blood flow is less stable. These are sites of arterial bifurcations and bending. Mass transfer between the plasma of bulk blood flow and the walls is greater at these sites. When the system of disposal and metabolism of LDL operates properly, the process of accumulation of LDL is very slow and these deposits do not produce a dangerous reduction of the lumen. When the system of disposal and metabolism of LDL deteriorates, the LDL accumulation on the vessel wall accelerates, and the difference between the norm and pathology (arteriosclerosis) is determined by the rate of deposition.

Mass transfer between the fluid and tube wall under pulsatile laminar flow is proportional to the flow velocity to the ⅓ power. This increases to the first power in the presence of transverse streamlines resulting from turbulent or locally unstable flows. Local instabilities appear as flow perturbations in the form of vortices, eddies and flow separation zones. Such instabilities are attributes of the pulsatile flow in the vicinity of small roughnesses or asperities on the wall and they are seen during certain phases of physiological blood flow. As the thickness of the deposits increases, the scope of the instabilities and the corresponding mass fluxes toward the vessel wall increase.

When a balance between the deposition and removal of LDL is disturbed in a particular location, the accumulation process accelerates in that location. A considerable lumen reduction, exacerbated by the retention of blood cells and calcium incrustation, results in self-accelerated LDL deposition.

Referring to FIG. 1, the variation of the relative blood flow velocity during one heartbeat is shown. As can be seen, the flow rate has a peak or maximum amplitude 10 during a first part 12 of the heartbeat. The flow rate decreases during a second part 14 of the heartbeat, and momentarily has a negative flow rate. During a third part 16 of the heartbeat, the flow rate settles out at a mean value. As can be seen in FIG. 1, the maximum flow velocity is about four times the mean velocity and, during a single heartbeat, the flow velocity is a damped and oscillating response.

With reference to FIG. 2, a schematic representation of flow velocity profiles at various moments within an artery 20 are illustrated. The artery 20 has walls 22, and the arrow indicates blood flow direction. The profiles 28 and 30 are of flattened form, and the profiles 24 and 30 have inflection points, which are indicators of flow instability (profile 28 has a pronounced flat portion 28′, while profile 26 has a pronounced sharp or pointed portion 26′). The profile 26 is stable for a short time. It is noted that the exact form of flow velocity profiles is difficult to reproduce among arteries and/or segments of one artery.

Upon entering a narrowed segment of an artery, or bypassing a wall roughness, blood flow accelerates, at least locally, thereby increasing the momentum and the kinetic energy of the flow. Substantial energy loss occurs in pulsatile flow at the exit of the narrow segment of the artery or downstream of the roughness, causing periods of turbulence and vortices at the boundaries of the artery. These phenomena increase with an increase in the magnitude of the stenosis, i.e. increase of the artery lumen area reduction, and in the magnitude of normal flow velocity through the artery.

With further reference to FIGS. 3A-3C, schematic representations of the flow pattern under three different arterial wall conditions are illustrated, each showing a two-dimensional projection on the longitudinal plane. FIG. 3A illustrates a flow within an artery 20 having smooth healthy walls 22. The velocity profile 32 is flattened because the flow is non-stationary.

FIG. 3B, which represents an early stage atheroma formation, illustrates a flow pattern within an artery 20 having one small wall roughness 34. As can be seen in FIG. 3B, most of the wall 22 is smooth, while the one small roughness 34 breaks the flow stability locally and sheds vortices 36 downstream from the roughness 34. Most of the velocity profile 32′ remains unchanged, and the length of the vortex sheet and the size of the vortices 36 differ at different moments and are difficult to reproduce.

FIG. 3C illustrates a developed atherosclerotic lesion 38, wherein wall thickening is considerable. Generally, such wall thickening is stretched and has an irregular shape. In the artery of FIG. 3C, the entire velocity profile 32″ is broken and the disturbance 40 is found across the entire arterial cross-section.

One imaging method for direct detection of arterial stenosis is angiography. This is an expensive and invasive imaging procedure that is not practical for routine screening. Non-invasive imaging methods for performing arterial screening include computerized axial tomography (CAT) and magnetic resonance imaging (MRI). These procedures use still framed images to determine whether a particular section of artery appears to be obstructed. While non-invasive, these procedures are costly and typically cannot be performed as part of a regular check up.

More recently, ultrasound imaging systems have been employed to detect and measure stenosis in the carotid artery by imaging the blood flow in the artery. Presently available ultrasound systems utilize the Doppler principle. In traditional Doppler ultrasound systems, a transducer directs a beam of ultrasound toward a blood vessel in which blood flow information is desired. Moving blood cells reflect ultrasound producing echoes, and either increase or decrease the frequency of the reflected sound depending on the velocity and direction of blood flow and the angle of incidence of the ultrasonic beam. More specifically, using a Doppler frequency shift, it is possible to recognize arterial flow disturbances, wherein erythrocytes, which are carried by the blood, serve as acoustical targets. The Doppler frequency shift is a function of the erythrocyte's velocity component that is parallel to a propagation vector of an acoustic beam generated by an ultrasound transducer. The greater the angle between the propagation vector of the acoustic beam and the erythrocyte velocity vector, the lower the Doppler frequency shift.

In continuous wave systems, a second transducer receives the echo and detects the frequency shift, from which velocity of the blood flow may be calculated. In pulse wave systems, a single transducer is used to direct the beam and receive the echo with a filter sorting out the signals to determine the frequency shift and hence the velocity of blood flow.

One limitation of Doppler ultrasound technology is that such systems can only measure the projection of the velocity parallel to the direction of the beam. If the beam is pointing at some angle with respect to the flow, the recorded velocity will be lower than the actual velocity to a degree proportional to the cosine of the angle of the beam with respect to the flow. To overcome this limitation, duplex Doppler ultrasound systems, which allow imaging to be used, along with traditional Doppler ultrasound systems, are used so that a region of interest may be “eye-balled” by an ultrasound technician and the beam may either be positioned at an appropriate angle or the angle of measurement may be recorded.

Another problem with the use of ultrasound systems is the presence of “noise” components in the Doppler shift frequency. The walls of blood vessels are dynamic in that they move in phase with a beating heart. During the systolic portion of the cardiac cycle the walls move out and during the diastolic portion the walls move in. These movements result in low and high frequency noise components returning with the echo of the Doppler signal.

Additionally, as blood flows through areas that have some degree of blockage, turbulence can be introduced in the blood flow in the form of vortices and eddy currents. The turbulence affects the blood flow and, therefore, the erythrocytes that are carried by the blood. Thus, the variability of the erythrocytes' trajectories due to turbulence in the blood flow and bending in the arteries adds to the power of the low frequency band of the Doppler spectrum.

Thus, the whole spectrum of Doppler frequency shift obtained from the arterial flow contains components both related and unrelated to a targeted disturbance. A targeted disturbance, as used herein, refers to a disturbance or disturbances caused by roughness or restriction in the artery, and not to disturbances caused by other factors or conditions, such as bending of the artery, the length of an artery segment or any other disturbance that is not a result of the atherosclerotic lesion.

For example, in straight, short segments of arteries the blood flow generally is not a fully developed pulsatile laminar flow, the overall spectral image is complex, and various local and temporal flow variations mask the targeted disturbances. These components can be characterized as noise and should be removed from the Doppler frequency shift and/or compensations made to the Doppler frequency shift in order to accurately ascertain the targeted disturbance.

Additionally, even without the noise components, targeted disturbances can be difficult to detect. For example, the magnitudes of the targeted disturbances can vary: 1) for different heartbeats; 2) with variations in the distance from the narrowest site of a stenosis; and/or 3) from individual to individual. Furthermore, the direction of insonation of the area of interest generally does not coincide with the direction of blood flow. As a result, the form of the velocity spectra produced by the Doppler echo is shifted from its true form, wherein the magnitude of the shift depends on the angular position of the transducer with respect to the blood flow.

The spectral image of the Doppler frequency shift also is influenced by individual factors affecting the blood flow in a specific sample, including, for example, cardiac performance, shape of the stenosis, compliance of the arterial wall and other factors.

In general, the prior art methods and devices rely upon visual imaging systems, which have difficulty forming images of the complex human anatomy, and require slow human visual analysis of each image. Additionally, noise components in the acquired signals make it difficult to accurately ascertain the location of a targeted disturbance.

Accordingly, there exists a need for a simple, inexpensive, non-invasive system and method of screening for arterial stenosis that does not rely upon visual imaging and is relatively unaffected by the shortcomings of previous systems. Additionally, it would be advantageous if such a system and method provides results that are individual insensitive. Furthermore, it would be advantageous if noise is separated or otherwise removed from a target signal, thereby substantially leaving a useful or meaningful signal.

### SUMMARY OF THE INVENTION

The present invention may be used, for example, in the examination of symptomatic patients, for monitoring disease development, for controlling treatment efficacy, and for screening of the asymptomatic aged population. The invention may be used for examination of the peripheral vascular system, such as, for example, the carotid arteries or the main branches of the abdominal aorta.

Another application area for the invention is the quantification of rapidly growing atheromas in the vicinity of a stent (which is a common complication in the surgical treatment of vascular diseases). The present invention also is applicable to the problem of accurately determining the blood flow pattern in the vicinity of the stent. This is useful, for example, for evaluating the quality of a stent implantation, the detection of undesirable cell growth or atheroma within the stent and for a prognosis of possible atheroma development inside or nearby the stent.

One aspect of the invention relates to a method of quantifying a degree of blockage in a vascular system, including: detecting Doppler shift data from a plurality of successive heartbeats, the Doppler shift data being obtained from arterial blood flow in an area of interest of the vascular system; transforming at least part of the Doppler shift data to the frequency domain; analyzing the transformed data with respect to individual-insensitive criteria; and calculating the degree of blockage based on the analyzed data.

Another aspect of the invention relates to a system for quantifying a degree of blockage in a vascular system, including: a processor circuit having a processor and a memory; and a measurement system stored in the memory and executable by the processor. The measurement system includes: logic that detects Doppler shift data from a plurality of successive heartbeats, the Doppler shift data being obtained from arterial blood flow in an area of interest of the vascular system; logic that transforms at least part of the Doppler shift data to the frequency domain; logic that analyzes the transformed data with respect to individual-insensitive criteria; and logic that calculates the degree of blockage based on the analyzed data.

Another aspect of the invention relates to a program embodied in a computer readable medium for quantifying a degree of blockage in a vascular system, including: code that detects Doppler shift data from a plurality of successive heartbeats, the Doppler shift data being obtained from arterial blood flow in an area of interest of the vascular system; code that transforms at least part of the Doppler shift data to the frequency domain; code that analyzes the transformed data with respect to individual-insensitive criteria; and code that calculates the degree of blockage based on the analyzed data.

To the accomplishment of the foregoing and related ends, the invention, then, comprises the features hereinafter fully described and particularly pointed out in the claims. The following description and the annexed drawings set forth in detail certain illustrative embodiments of the invention. These embodiments are indicative, however, of but a few of the various ways in which the principles of the invention may be employed. Other objects, advantages and novel features of the invention will become apparent from the following detailed description of the invention when considered in conjunction with the drawings.

### BRIEF DESCRIPTION OF THE DRAWINGS

These and further features of the present invention will be apparent with reference to the following description and drawings, wherein:

FIG. 1 is a graphical representation of the physiological blood flow;

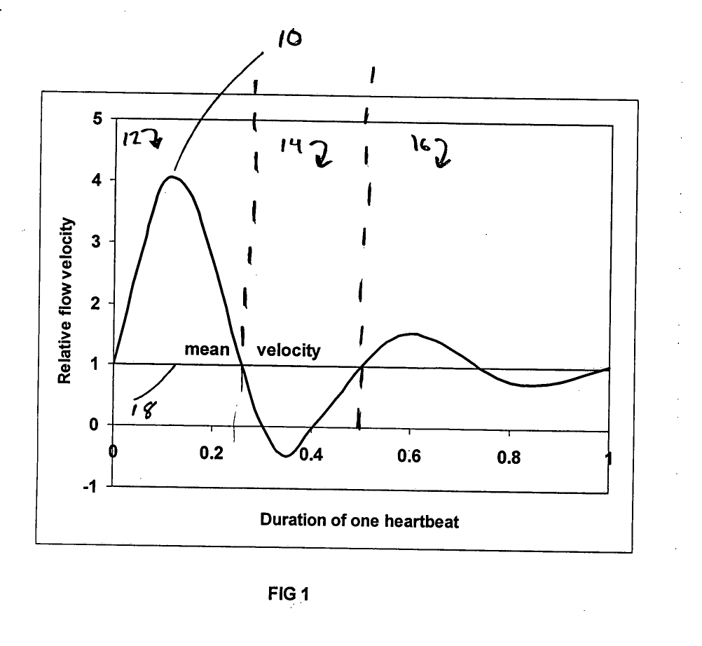

FIG. 2 is a plot of flow velocity profiles at various moments;

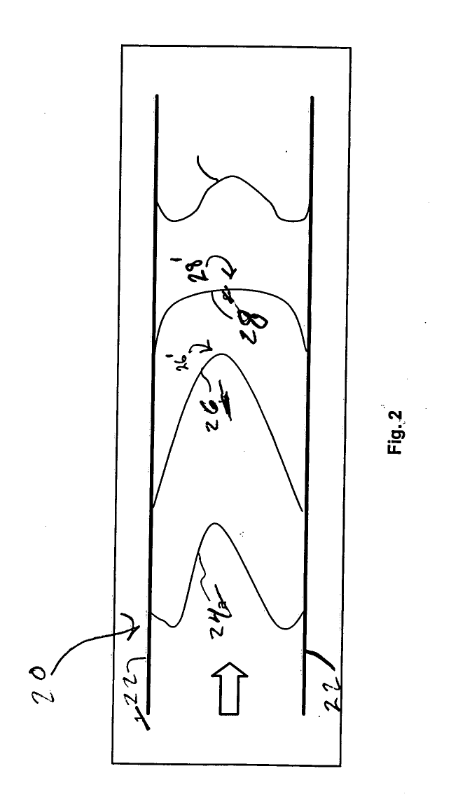

FIGS. 3A-3C are schematic representations of the flow pattern under three different arterial wall conditions;

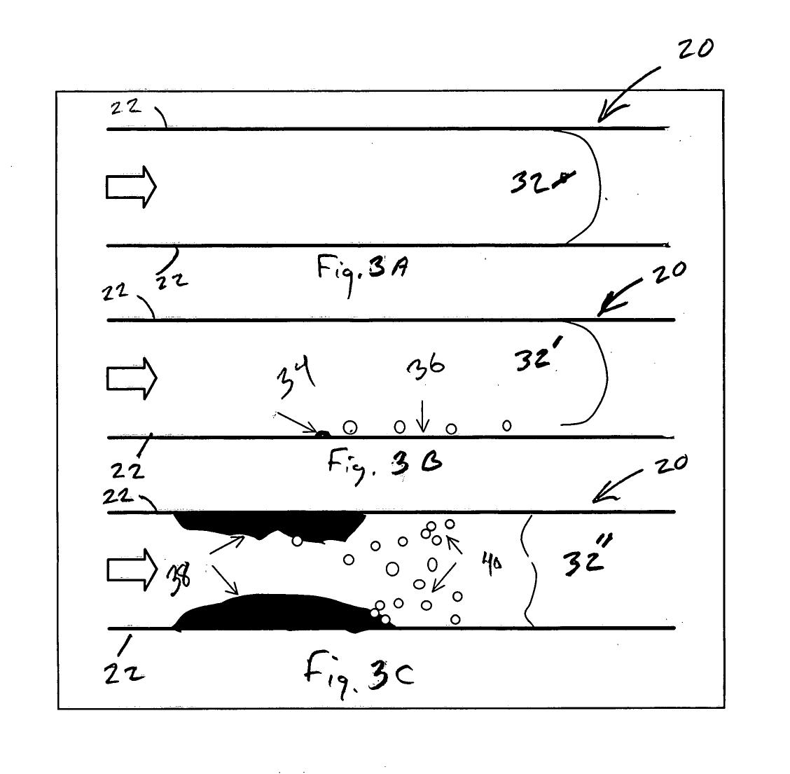

FIG. 4 is a schematic diagram of a system in accordance with an embodiment of the present invention;

FIG. 5 is a plot of the velocity spectrum (or Doppler frequency shifts) versus the fraction or phase of one heart cycle in the time domain;

FIG. 6 is a plot of a selection in the frequency domain of the necessary frequency band and of its width in accordance with an embodiment of the present invention;

FIG. 7 is a plot of Equation 2 over a range of possible arterial lumen reduction in accordance with an embodiment of the present invention;

FIG. 8 is a plot of the linearized relation between the logarithm of the calculated value and the percent of lumen area reduction in accordance with an embodiment of the present invention;

FIG. 9 is a flow chart of a method of quantifying arterial stenosis in accordance with an embodiment of the present invention;

FIG. 10 is a flow chart of a method of quantifying arterial stenosis in accordance with another embodiment of the present invention; and

FIG. 11 illustrates an exemplary computer system used to implement the present invention.

### DESCRIPTION OF THE INVENTION

The following is a description of the present invention with reference to the attached drawings, wherein like reference numerals will refer to like elements throughout. To illustrate the present invention in a clear and concise manner, the drawings may not necessarily be to scale. Additionally, features that are described and/or illustrated with respect to one embodiment may be used in the same way or in a similar way in one or more other embodiments and/or in combination with or instead of the features of other embodiments.

The present invention relates to a system and method for quantifying a level of arterial stenosis, i.e., arterial lumen area reduction in a vascular system, and will be described with respect to a human vascular system. It should be appreciated, however, that the invention also can be applied to non-human vascular systems, e.g., animal vascular systems.

According to an embodiment of the invention, Doppler shift data from multiple heartbeats are used to determine a broad range of local and temporal variations in flow disturbances in an area of interest, e.g., an area where wall thickening is suspected. Selected portions of the Doppler shift data are transformed from the time domain to the frequency domain, and the frequency band is analyzed. More specifically, the transformed data is analyzed with respect to individual-insensitive criteria and, based on the analysis, the level or amount of lumen area reduction is calculated and provided to medical personnel. As used herein, individual insensitive criteria refers to criteria that provides substantially the same results regardless who performs the procedure and/or who the procedure is performed on. For example, heart performance and/or vessel anatomy differ among individuals, and these differences influence the peculiarities of Doppler spectra. Using individual-insensitive criteria, such influences are minimized or eliminated.

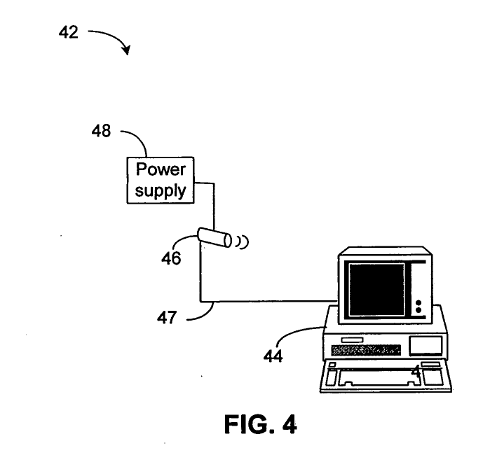

With reference to FIG. 4, a system 42 that can be used to implement the present invention is illustrated. The system 42 includes a computer system 44 for executing logic, such as instructions or code, and an ultrasonic transducer 46 communicatively coupled to the computer system 44. The ultrasonic transducer 46 can be coupled to the computer system 44 via a communication link 47, such as a wired link or a wireless link, e.g., 802.11a, b, g, Bluetooth, or the like. A power supply 48 provides power to the ultrasonic transducer to operate the transducer.

Depending on the target characteristics (e.g. depth) and design specifics, the transducer 46 can operate in a frequency band ranging from 2.5 to 10 MHz. Additionally, the transducer 46 can include a single ultrasonic unit operating in a wide frequency range, or it can include a set of separate interchangeable ultrasonic units that operate in a specific frequency range. A sender and receiver (not shown) can be mounted within each unit. The transducer 46 can operate in the pulsed width mode, continuous wave mode, or any other mode that provides satisfactory imaging of the vascular system. The transducer 46 may be suitable to perform duplex scanning (e.g., Doppler and B-scan, if necessary to find the stenosis).

According to the present invention, ultrasound at a certain carrier frequency insonifies the area of interest, e.g., the targeted disturbance or region of arterial wall reduction. Conventional scanning techniques can be used to search the area of interest for stenosis. Conventional techniques can include, for example, combining continuous wave insonation (CW-mode) for gray scale, B-scan imaging of the target with pulsed wave (PW-mode) insonation and reception of Doppler shifted echoes, and the like. As was noted above, the operational frequency of insonation can range, for example, between about 2.5 to 10 MHz. The optimum frequency depends on the depth of immersion of the artery into surrounding tissues, e.g., on the distance from transducer to the targeted vessel, on the depth of the targeted disturbance and on the sampling volume being examined. The depth or “reach” of ultrasound propagation depends on the distance and frequency; the greater the distance the lower the frequency.

If the position of stenosis is already known and there are no other vessels in the vicinity of the area of interest, a continuous wave (CW) mode can be used for obtaining the Doppler signal. If, on the other hand, the position of the stenosis is not known, a duplex scanning technique can be applied. For example, a gray scale B-scan image can be produced via CW insonation, and the gray scale B-scan image can be used to position a pulsed wave (PW) beam in the area of interest. Preferably, a cross-section of the target vessel is entirely within the sampled volume of the B-scan image.

As was noted above, erythrocytes in the blood serve as acoustical targets. The Doppler frequency shift of the echo signals from the moving erythrocytes may vary from 100 to 11,000 Hz, with the usual range being about 3000 Hz. The returning echoes from the insonation are recorded for later use.

Once the Doppler frequency shifts are recorded, they are transformed from the time domain to the frequency domain using a Fast Fourier Transform or a Wavelet transform, for example. More specifically, a transformation is performed by a computer on a selected part of the time domain data, and the Doppler shift at a certain point in the cardiac cycle is determined in the frequency domain. The selected part of the time domain data can be an interval at the beginning of systole, for example.

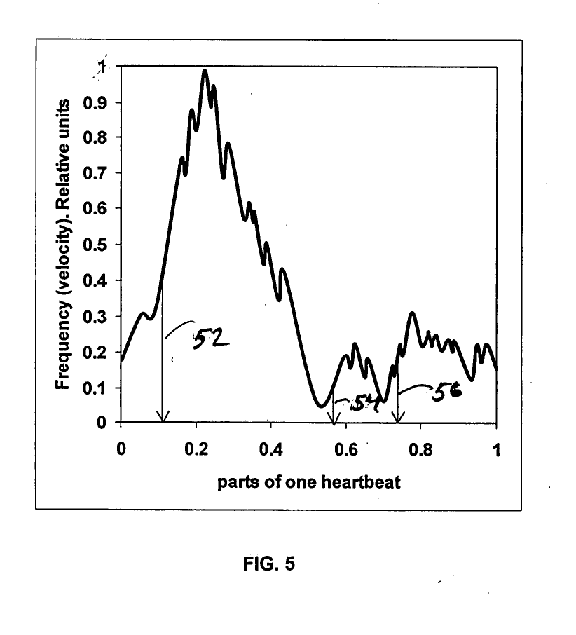

FIG. 5 is a plot of the velocity spectrum (or Doppler frequency shifts) versus the fraction or phase of one heart cycle in the time domain. Arrows 52, 54, 56 indicate phase points around which transformations into the frequency domain may be made, e.g., approximately in the first one-third of an accelerated flow region.

The exact timing or part of the time domain data where the transformation is performed, however, is not critical due to the variations produced by successive heartbeats. Other points in time during which a flow is accelerating also can be used.

Once the phase points are transformed to the frequency domain, the maximum amplitude of the Doppler shift is identified and measured for this frequency domain spectnum. The maximum amplitude then is applied to each of a predetermined number of heartbeat spectra, as described below.

In applying the maximum amplitude to each of the plurality of heartbeat spectra, a pre-selected parameter characterizing a shape of the frequency band is measured at a predetermined fraction of the spectrum's maximum amplitude. According to one embodiment, the pre-selected parameter is a spectrum bandwidth of the transformed phase points in the frequency domain. Preferably, the fractional amount is between 0.1 and 0.9, more preferably between 0.25 and 0.75 and even more preferably between 0.4 and 0.6. In one embodiment, half or 0.5 of the maximum amplitude is used to measure the pre-selected parameter. A small fraction, such as 0.25, is more informative but highly sensitive to instrument noise. A larger fraction, such as 0.75, is noise tolerant, but does not capture the necessary spectral peculiarities as well as the smaller fractions. Thus, a good compromise is achieved in the range of 0.4-0.6. For example, the measured amplitude of the spectrum is multiplied by a selected fraction, e.g., 0.5, to arrive at a fractional amount of the measure spectrum. Using the fractional amount of the measured spectrum, the pre-selected parameter is identified in a graph of the frequency band, as is described below.

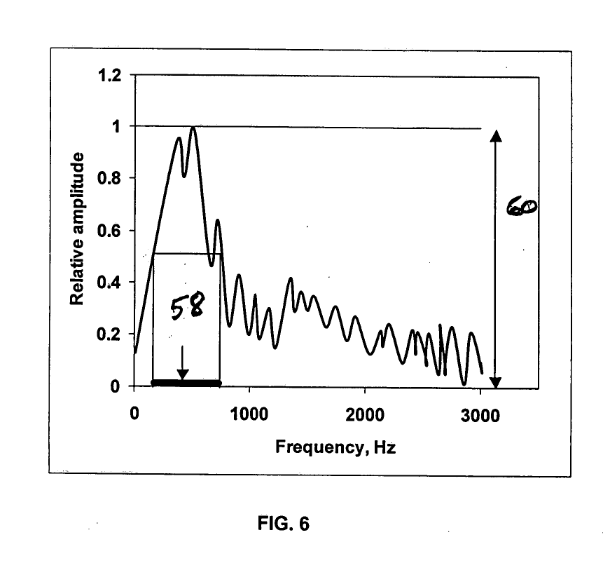

FIG. 6 illustrates the selection for an exemplary one of the phase points 52, 54 or 56 in the frequency domain of the necessary frequency band and of its spectrum bandwidth 58. The abscissa values are Fourier Transform frequencies, and the ordinate values are normalized Fourier Transform amplitudes. The level of measurement in this example is selected at one-half of the maximum amplitude 60. The power of this frequency band (FIG. 6), which is associated with the maximum amplitude, increases when trajectories of insonified erythrocytes significantly differ from the acoustic propagation vector, since the greater the angle between the acoustical beam and the erythrocyte velocity vector, the lower the cosine of this angle and the lower the Doppler shift frequency. This Fourier Transform measurement approach represents an indirect characterization of the power of the selected low-frequency band of the Doppler spectrum (FIG. 5).

In contrast to the present invention, the prior art methods of directly evaluating the power of the selected frequency band or using other possible indices provide individual-sensitive results that are very sensitive to the angular positioning of the transducer. The Fourier transform measurement approach according to the present invention provides results that are individual-insensitive, less dependent on the transducer positioning and, therefore, more applicable for practical diagnostics.

According to an embodiment of the invention, the measurement of the spectrum bandwidth is repeated for spectra from at least three successive heartbeats at several points along an arterial segment that is under examination, e.g., along about a 1.0 to about 1.5 cm segment of the area of interest. The maximum measured value of the pre-selected parameter determined as a result of evaluations of all sampled heartbeats at all points is used to calculate the percentage of lumen reduction, as described below.

$$LR = 100 \% * ( 1 - \frac{{r}^{2}}{{R}^{2}} ) \qquad Equation 1$$

Equation 1 expresses the percentage of lumen reduction (LR), where R is the radius (in mm) of a healthy artery and r is the minimal lumen radius (in mm) within a stenosis. If the spectrum bandwidth 58 (e.g., the spectral width at the fraction of the maximum amplitude), determined as described above, is used as the pre-selected parameter, then a linear relation exists between the actual LR and the natural logarithm of the spectrum bandwidth value. For example, for the internal carotid artery examined with the pulsed wave (PW) Doppler mode with 10 MHz carrier frequency, the relationship between the spectrum bandwidth (BW) 58 of the early systole at the level of one half (0.5) of the maximum amplitude and LR is expressed by Equation 2 below. As will be appreciated by those skilled in the art, the slope term (124.9) of Equation 2 changes as different fractional amplitudes are used to determine the spectrum bandwidth BW.
LR=39.22*In(BW)−124.9 Equation 2

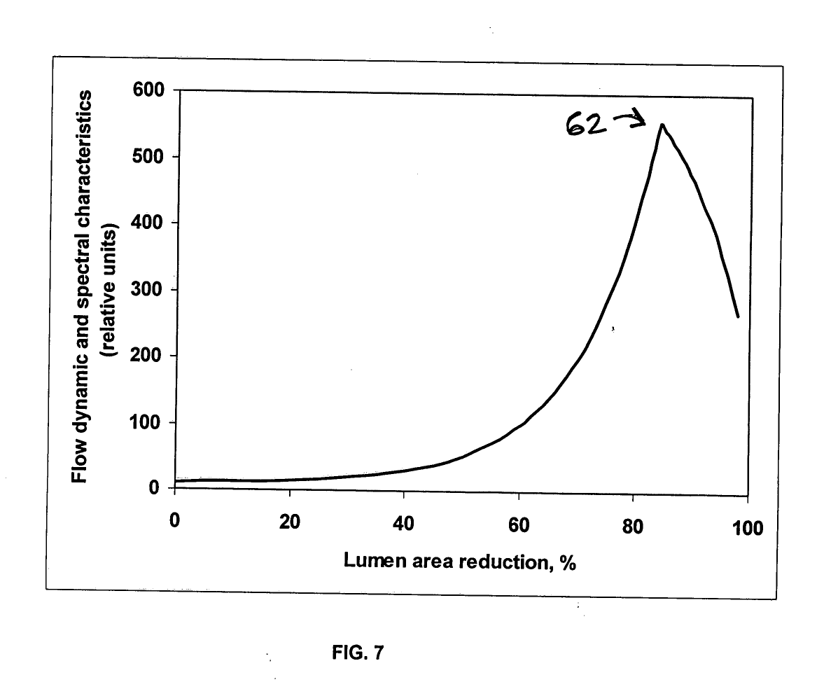

FIG. 7 is a plot of Equation 2 over the whole range of possible arterial lumen reduction, where the abscissa is the lumen area reduction (0 to 100%), and the ordinate is a spectral characterization parameter. This function has an inflection point 62 in the area of 80% to 90% of lumen reduction. When the degree of stenosis is greater than 70% of the lumen area, the same calculated spectral parameter may be obtained for stenoses in the ranges of 70% to 85% and 85% to 98%. The systolic maximum flow velocity and flow pressure gradient have a peak in the same area of 80% to 90% of lumen area reduction. Thus, severe stenoses simultaneously affect the dynamic and spectral characteristics of the flow. Accordingly, stenoses having greater than 85% blockage may not be reliably identified using Equation 2.

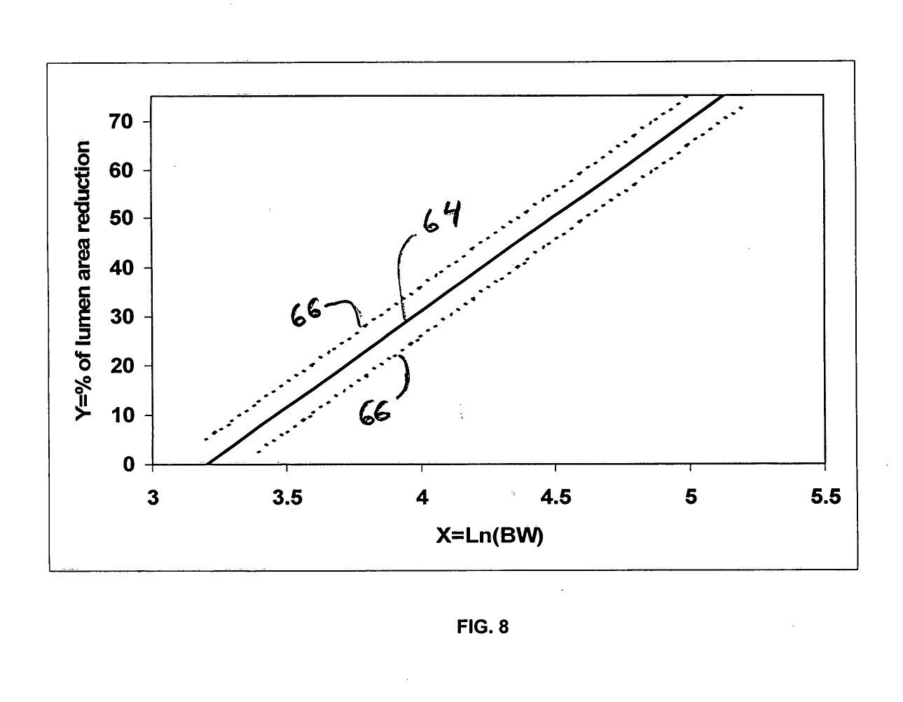

FIG. 8 is a plot of the linearized relation between the logarithm of the spectrum bandwidth, e.g., the width 58 of the selected frequency band, and the percent of lumen area reduction. Here, the abscissa is the logarithm of the calculated pre-selected value in the chosen low-frequency band, and the ordinate is the percent of lumen area reduction. This relation is accurate for the 10% to 70% range of lumen area reduction. The solid line 64 indicates the best fit, and dotted lines 66 are the limits of the standard error.

The insonation frequency and the angular orientation of the transducer can be changed in the course of scanning the area of interest, depending on the depth of the target and on any change of direction of the vessel's axis. Both the insonation frequency and the angular orientation influence the peak frequency (Peak frequency is the frequency of maximal amplitude) and In(BW) in the frequency domain. Equation 3 below captures this relationship and, when used in Equation 4, provides a robust, operator-independent mechanism for determining lumen area reduction LR, while being more tolerant of poor signal quality than using Equation 2 alone. In the following equations, BW is the spectrum bandwidth of the early systole at the level of one half of the maximum amplitude and “rec” indicates recorded values of BW and peak frequency. As was noted above with respect to Equation 2, the slope term (2.1726) of Equation 3 changes as different fractional amplitudes are used to determine BW, e.g., the slope increases when a predetermined fraction less that 0.5 is used.

$$\begin{matrix}ln ( BW ) = 0.791 * ln ( PeakFrequency ) + 2.1726 & Equation 3 \\ ln ( BWstan dard ) = 3.715 * \frac{ln ( {BW}_{rec} )}{0.791 * ln ( {PeakFrequency}_{rec} ) + 2.1726} & Equation 4\end{matrix}$$

Equation 4 can be used in conjunction with Equation 2 to provide improved results for the calculation of the lumen reduction LR, as is described in more detail below.

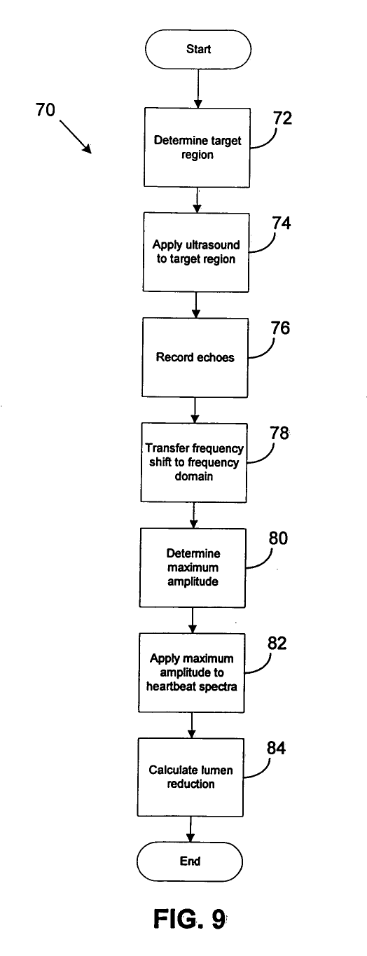

Moving now to FIG. 9, a flow diagram 70 illustrating the method of determining the level of stenosis within the vascular system in accordance with an embodiment of the invention is shown. The flow diagram includes a number of process blocks arranged in a particular order. As should be appreciated, many alternatives and equivalents to the illustrated steps may exist and such alternatives and equivalents are intended to fall with the scope of the claims appended hereto. Alternatives may involve carrying out additional steps or actions not specifically recited and/or shown, carrying out steps or actions in a different order from that recited and/or shown, and/or omitting recited and/or shown steps. Alternatives also include carrying out steps or actions concurrently or with partial concurrence.

Beginning at step 72, the area of interest or “targeted disturbance” is determined using conventional techniques. For example, CW-mode and/or B-scan imaging can be used to search and locate for the area of interest. Once the area of interest is identified, the area is scanned using ultrasonic waves and the echoes are recorded, as indicated at steps 74 and 76. The echoes provide data relating to the blood flow disturbances in the area of interest (the Doppler frequency shifts). More specifically, the data obtained from the scan is plotted as shown in FIG. 5 to obtain the velocity spectrum over one heart cycle.

At step 78, a portion of the recorded data is transformed from the time domain to the frequency domain using a Fast Fourier transform, for example. More specifically, subintervals of time domain data most suitable for analysis in the frequency domain are selected from each time domain spectra, such as areas during which a flow is accelerating, for example. The spectra corresponding to the subintervals can be digitized and stored in memory of a computer system.

Next, the maximum amplitude of the Doppler shift is identified and measured in the frequency domain, and the maximum amplitude is applied to the heartbeat spectra, as indicated at steps 80 and 82. More specifically, a fraction of the maximum amplitude is used to determine the value of the spectrum bandwidth. For example, and with reference to FIG. 6, the spectrum bandwidth 58 (e.g., the pre-selected parameter) is determined at a fraction of the maximum amplitude 60 (0.5 of maximum amplitude), and the value of the spectrum width 58 is recorded. This measurement is repeated for a number of consecutive heartbeats along several points of the arterial segment, and a maximum value of the spectrum bandwidth is identified. At step 84, the maximum value of the spectrum bandwidth is used in Equation 2 (BW) to estimate the lumen reduction, and the result is reported to the user, e.g., it is displayed on an operator display.

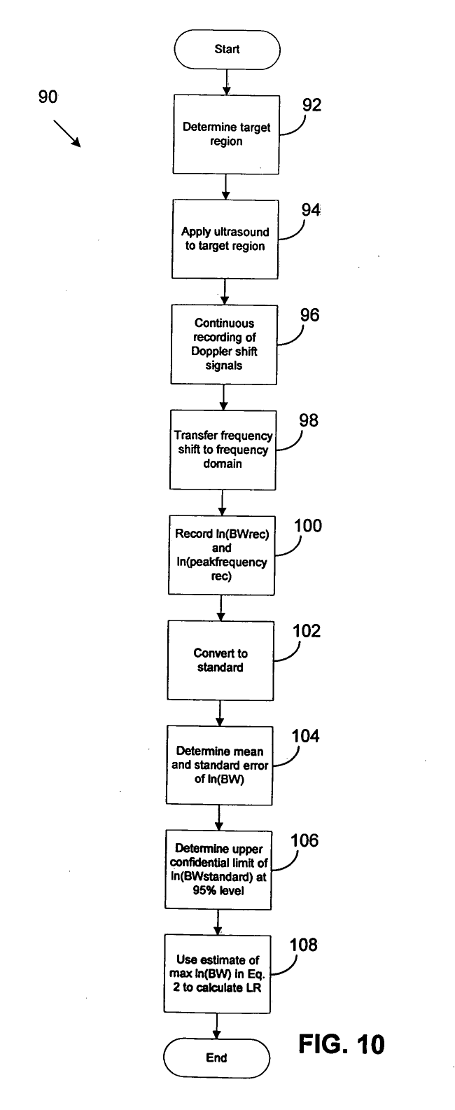

With reference now to FIG. 10, a flow diagram 90 illustrating the method of determining the level of stenosis within the vascular system in accordance with another embodiment of the invention is shown. Beginning at step 92, the area of interest is determined using conventional techniques as described previously. Once the area of interest is identified, the area is scanned using ultrasonic waves and the echoes are recorded, as indicated at step 94.

Moving to step 96, Doppler shift signals are continuously recorded during a specified period, e.g., a five second period to about a fifteen second period and at step 98, a Fourier transform is performed on the recorded signals in a succession of continuous intervals. It is preferable that the intervals be about one-half (0.5) seconds each. For example, if Doppler shift signals are continuously recorded for a five second period, then ten intervals are used to obtain 0.5 seconds/interval (5 sec/10 intervals). Similarly, if the Doppler shift signals are recorded for a fifteen second period, then thirty intervals are used to obtain 0.5 seconds/interval. The number of 0.5 second intervals can be as few as ten, more preferably about fifteen, and even more preferably thirty.

At step 100, values for In(BW_{rec}) and In(PeakFrequency_{rec}) are recorded, e.g., stored in computer memory. The number “n” of recorded values can be any number of points. The recorded values of In(BW_{rec}) are converted to standard form (In(BW_{standard})) using Equation 4, and the mean <In(BW_{standard})> and standard error (SE) of In(BW_{standard}) for the plurality of recorded values are calculated using conventional methods, as indicated at steps 102 and 104.
UCL=<In(BW_{standard})>+2.26*SE Equation 5

Moving to step 106, the upper confidential limit (UCL) of the calculated mean value (<In(BW_{standard})> at the 95% level is determined using Equation 5, wherein 2.26 is the Student's criterion for 10−1=9 degrees of freedom. If the number of recorded points n differs from ten, the value of Student's criterion should be taken for n−1 degrees of freedom. The UCL (the estimate of the maximum In(BW_{standard})) is placed in Equation 2 to calculate the percent lumen area reduction LR, as indicated at step 108.

Statistical analysis of experimental data has confirmed that the resulting LR estimate is individual-insensitive, has the standard error of 4.65% and is applicable to stenosis in the range of 10% to 70%. The same formula can be extended to estimate LR in range of 70% to 80%. In this higher range, however, results can be uncertain because the value of the function declines as it passes through a peak within the interval of LR=80 to 90%.

During an actual patient examination, the calculated LR values can be presented on a computer display. The described procedure does not require special operator skills, and is more tolerant to variations of angular positioning of the transducer then prior art methods. This procedure is also more tolerant of variations in the carrier frequency of insonation, but it needs a somewhat longer continuous record.

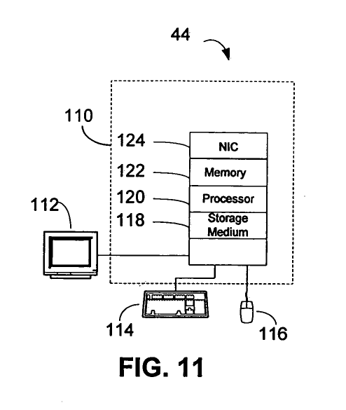

Moving to FIG. 11, a computer system 44 for executing a computer program in accordance with the present invention is illustrated. The computer system 44 includes a computer 110 for processing data, and a display 112 for viewing system information. The technology used in the display is not critical and may be any type currently available, such as a flat panel liquid crystal display (LCD) or a cathode ray tube (CRT) display, or any display subsequently developed. A keyboard 114 and pointing device 116 may be used for data entry, data display, screen navigation, etc. The keyboard 114 and pointing device 116 may be separate from the computer 110 or they may be integral to it. A computer mouse or other device that points to or otherwise identifies a location, action, etc., e.g., by a point and click method or some other method, are examples of a pointing device. Alternatively, a touch screen (not shown) may be used in place of the keyboard 114 and pointing device 116. A touch screen is well known by those skilled in the art and will not be described in detail herein. Briefly, a touch screen implements a thin transparent membrane over the viewing area of the display 112. Touching the viewing area sends a signal to the computer 110 indicative of the location touched on the screen. The computer 110 may equate the signal in a manner equivalent to a pointing device and act accordingly. For example, an object on the display 112 may be designated in software as having a particular function (e.g., view a different screen). Touching the object may have the same effect as directing the pointing device 116 over the object and selecting the object with the pointing device, e.g., by clicking a mouse. Touch screens may be beneficial when the available space for a keyboard 114 and/or a pointing device 116 is limited.

Included in the computer 110 is a storage medium 118 for storing information, such as application data, screen information, programs, etc. The storage medium 118 may be a hard drive, for example. A processor 120, such as an AMD Athlon 64™ processor or an Intel Pentium IV® processor, combined with a memory 122 and the storage medium 118 execute programs to perform various functions, such as data entry, numerical calculations, screen display, system setup, etc. A network interface card (NIC) 114 allows the computer 110 to communicate with devices external to the computer system 4.

The actual code for performing the functions described herein can be readily programmed by a person having ordinary skill in the art of computer programming in any of a number of conventional programming languages based on the disclosure herein. Consequently, further detail as to the particular code itself has been omitted for sake of brevity. As will be appreciated, the various computer codes for carrying out the processes herein described can be embodied in computer-readable media.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A method of quantifying a degree of blockage in a vascular system, comprising:
detecting Doppler shift data from a plurality of successive heartbeats, the Doppler shift data being obtained from arterial blood flow in an area of interest of the vascular system;
transforming at least part of the Doppler shift data to the frequency domain;
analyzing the transformed data with respect to individual-insensitive criteria; and
calculating the degree of blockage based on the analyzed data.

2. The method of claim 1, wherein detecting Doppler shift data from a plurality of successive heartbeats includes using at least three successive heartbeats at several points along the area of interest.

3. The method of claim 1, wherein transforming the data into the frequency domain includes using a Fast Fourier Transform or a Wavelet transform on the Doppler shift data.

4. The method of claim 1, wherein transforming at least part of the Doppler shift data includes selecting part of the Doppler shift data that corresponds to an interval of a cardiac cycle.

5. The method of claim 4, wherein selecting part of the Doppler shift data includes selecting at least one of an interval at the beginning of systole or an interval in which a flow is accelerating.

6. The method of claim 1, wherein analyzing the transformed data with respect to individual-insensitive criteria includes:
identifying a maximum amplitude of the Doppler shift data; and
using the maximum amplitude to measure a pre-selected parameter.

7. The method of claim 6, wherein using the maximum amplitude includes using a predetermined fraction of the maximum amplitude to measure the pre-selected parameter.

8. The method of claim 7, wherein using a predetermined fraction includes using a predetermined fraction between about 0.1 and 0.9 of the maximum amplitude.

9. The method of claim 6, wherein using the maximum amplitude to measure a pre-selected parameter includes using a spectrum bandwidth as the pre-selected parameter.

10. The method of claim 9, wherein calculating the degree of blockage includes using the measured spectrum bandwidth as an independent variable in a function that determines the level of blockage.

11. The method of claim 10, wherein using the measured spectrum bandwidth as an independent variable in a function includes using a function given by LR=39.22*In(BW)−124.9, wherein LR is the percent lumen reduction and BW is the maximum measured spectrum bandwidth (Hz) at one half the maximum amplitude.

12. The method of claim 1, wherein calculating the degree of blockage includes:
calculating a spectrum bandwidth from recorded values of the transformed waveform; and
using the calculated spectrum bandwidth as an independent variable in a function that determines the level of blockage.

13. The method of claim 12, wherein calculating the spectrum bandwidth includes using a function given by
In(BW_{standard})=3.715*In(BW_{rec})/(0.791*In(PeakFrequency_{rec})+2.1726), wherein BW_{standard} is the standard spectrum bandwidth (Hz) at early systole, BW_{rec} is a recorded spectrum bandwidth (Hz) at early systole, and PeakFrequency_{rec} is a recorded maximum frequency (Hz) of the transformed waveform.

14. The method of claim 13, wherein calculating the spectrum bandwidth includes using a function given UCL=<In(BW_{standard})>+2.26*SE, wherein UCL is a calculated upper confidential limit (Hz), <In(BWstandard)> is a calculated mean value of In(BW_{standard}) (Hz), and SE is a calculated standard error of In(BW_{standard}) (Hz).

15. The method of claim 14, wherein using the calculated spectrum bandwidth as an independent variable in a function includes using a function given by LR=39.22*In(BW)−124.9, wherein LR is the percent lumen reduction and BW is the calculated UCL (Hz).

16. The method of claim 1, wherein detecting Doppler shift data includes continuously recording Doppler shift data during a specified period.

17. The method of claim 16, wherein transforming at least part of the Doppler shift data includes performing transforms in a continuous succession of continuous intervals.

18. A system for quantifying a degree of blockage in a vascular system, comprising:
a processor circuit having a processor and a memory;
a measurement system stored in the memory and executable by the processor, the measurement system comprising:
logic that detects Doppler shift data from a plurality of successive heartbeats, the Doppler shift data being obtained from arterial blood flow in an area of interest of the vascular system;
logic that transforms at least part of the Doppler shift data to the frequency domain;
logic that analyzes the transformed data with respect to individual-insensitive criteria; and
logic that calculates the degree of blockage based on the analyzed data.

19. The system of claim 18, wherein the logic that detects Doppler shift data from a plurality of successive heartbeats includes logic that uses at least three successive heartbeats at several points along the area of interest.

20. The system of claim 18, wherein the logic that transforms the data into the frequency domain includes logic that uses a Fast Fourier Transform or a Wavelet transform on the Doppler shift data.

21. The system of claim 18, wherein the logic that transforms at least part of the Doppler shift data includes logic that selects part of the Doppler shift data that corresponds to an interval of a cardiac cycle.

22. The system of claim 21, wherein the logic that selects part of the Doppler shift data includes logic that selects at least one of an interval at the beginning of systole or an interval in which a flow is accelerating.

23. The system of claim 18, wherein the logic that analyzes the transformed data with respect to individual-insensitive criteria includes:
logic that identifies a maximum amplitude of the Doppler shift data; and
logic that uses the maximum amplitude to measure a pre-selected parameter.

24. The system of claim 23, wherein the logic that uses the maximum amplitude includes logic that uses a predetermined fraction of the maximum amplitude to measure the pre-selected parameter.

25. The system of claim 24, wherein the logic that uses a predetermined fraction includes logic that uses a predetermined fraction between about 0.1 and 0.9 of the maximum amplitude.

26. The system of claim 23, wherein the logic that uses the maximum amplitude to measure a pre-selected parameter includes logic that uses a spectrum bandwidth as the pre-selected parameter.

27. The system of claim 26, wherein the logic that calculates the degree of blockage includes logic that uses the measured spectrum bandwidth as an independent variable in a function that determines the level of blockage.

28. The system of claim 27, wherein the logic that uses the measured spectrum bandwidth as an independent variable in a function includes logic that uses a function given by LR=39.22*In(BW)−124.9, wherein LR is the percent lumen reduction and BW is the maximum measured spectrum bandwidth (Hz) at one half the maximum amplitude.

29. The system of claim 18, wherein the logic that calculates the degree of blockage includes:
logic that calculates a spectrum bandwidth from recorded values of the transformed waveform; and
logic that uses the calculated spectrum bandwidth as an independent variable in a function that determines the level of blockage.

30. The system of claim 29, wherein the logic that calculates the spectrum bandwidth includes logic that uses a function given by
In(BW_{standard})=3.715*In(BW_{rec})/(0.791*In(PeakFrequency_{rec})+2.1726), wherein BW_{standard} is the standard spectrum bandwidth (Hz) at early systole, BW_{rec} is a recorded spectrum bandwidth (Hz) at early systole, and PeakFrequency_{rec} is a recorded maximum frequency (Hz) of the transformed waveform.

31. The system of claim 30, wherein the logic that calculates the spectrum bandwidth includes logic that uses a function given UCL=<In(BW_{standard})>+2.26*SE, wherein UCL is a calculated upper confidential limit (Hz), <In(BWstandard)> is a calculated mean value of In(BW_{standard}) (Hz), and SE is a calculated standard error of In(BW_{standard}) (Hz).

32. The system of claim 31, wherein the logic that uses the calculated spectrum bandwidth as an independent variable in a function includes logic that uses a function given by LR=39.22*In(BW)−124.9, wherein LR is the percent lumen reduction and BW is the calculated UCL (Hz).

33. The system of claim 18, wherein the logic that detects Doppler shift data includes logic that continuously records Doppler shift data during a specified period.

34. The system of claim 33, wherein the logic that transforms at least part of the Doppler shift data includes logic that performs transforms in a continuous succession of continuous intervals.

35. A program embodied in a computer readable medium for quantifying a degree of blockage in a vascular system, comprising:
code that detects Doppler shift data from a plurality of successive heartbeats, the Doppler shift data being obtained from arterial blood flow in an area of interest of the vascular system;
code that transforms at least part of the Doppler shift data to the frequency domain;
code that analyzes the transformed data with respect to individual-insensitive criteria; and
code that calculates the degree of blockage based on the analyzed data.

36. The program of claim 35, wherein the code that detects Doppler shift data from a plurality of successive heartbeats includes code that uses at least three successive heartbeats at several points along the area of interest.

37. The program of claim 35, wherein the code that transforms the data into the frequency domain includes code that uses a Fast Fourier Transform or a Wavelet transform on the Doppler shift data.

38. The program of claim 35, wherein the code that transforms at least part of the Doppler shift data includes code that selects part of the Doppler shift data that corresponds to an interval of a cardiac cycle.

39. The program of claim 38, wherein the code that selects part of the Doppler shift data includes code that selects at least one of an interval at the beginning of systole or an interval in which a flow is accelerating.

40. The program of claim 35, wherein the code that analyzes the transformed data with respect to individual-insensitive criteria includes:
code that identifies a maximum amplitude of the Doppler shift data; and
code that uses the maximum amplitude to measure a pre-selected parameter.

41. The program of claim 40, wherein the code that uses the maximum amplitude includes code that uses a predetermined fraction of the maximum amplitude to measure the pre-selected parameter.

42. The program of claim 41, wherein the code that uses a predetermined fraction includes code that uses a predetermined fraction between about 0.1 and 0.9 of the maximum amplitude.

43. The program of claim 40, wherein the code that uses the maximum amplitude to measure a pre-selected parameter includes code that uses a spectrum bandwidth as the pre-selected parameter.

44. The program of claim 43, wherein the code that calculates the degree of blockage includes code that uses the measured spectrum bandwidth as an independent variable in a function that determines the level of blockage.

45. The program of claim 44, wherein the code that uses the measured spectrum bandwidth as an independent variable in a function includes code that uses a function given by LR=39.22*In(BW)−124.9, wherein LR is the percent lumen reduction and BW is the maximum measured spectrum bandwidth (Hz) at one half the maximum amplitude.

46. The program of claim 35, wherein the code that calculates the degree of blockage includes:
code that calculates a spectrum bandwidth from recorded values of the transformed waveform; and
code that uses the calculated spectrum bandwidth as an independent variable in a function that determines the level of blockage.

47. The program of claim 46, wherein the code that calculates the spectrum bandwidth includes code that uses a function given by In(BW_{standard})=3.715*In(BW_{rec})/(0.791 *In(PeakFrequency_{rec})+2.1726), wherein BW_{standard} is the standard spectrum bandwidth (Hz) at early systole, BW_{rec} is a recorded spectrum bandwidth (Hz) at early systole, and PeakFrequency_{rec} is a recorded maximum frequency (Hz) of the transformed waveform.

48. The program of claim 47, wherein the code that calculates the spectrum bandwidth includes code that uses a function given UCL =<In(BW_{standard})>+2.26*SE, wherein UCL is a calculated upper confidential limit (Hz), <In(BWstandard)>is a calculated mean value of In(BW_{standard}) (Hz), and SE is a calculated standard error of In(BW_{standard}) (Hz).

49. The program of claim 48, wherein the code that uses the calculated spectrum bandwidth as an independent variable in a function includes code that uses a function given by LR=39.22*In(BW)−124.9, wherein LR is the percent lumen reduction and BW is the calculated UCL (Hz).

50. The program of claim 35, wherein the code that detects Doppler shift data includes code that continuously records Doppler shift data during a specified period.

51. The program of claim 50, wherein the code that transforms at least part of the Doppler shift data includes code that performs transforms in a continuous succession of continuous intervals.
